(19) United States
(12) Patent Application Publication
(10) Pub. No.: US 2008/0312560 A1
(43) Pub. Date: Dec. 18, 2008

(54) Transducer Unit, Device Arrangement and a Method Utilizing the Device Arrangement for Creating and Presenting an Estimate of Bone Mass Development

(75) Inventors: Ari Jamsen, Oulu (FI); Anneli Rinta-Paavola, Oulu (FI); Raija Korpelainen, Oulu (FI); Timo Jamsa, Oulu (FI); Juhani Leppaluoto, Oulu (FI)

Correspondence Address: VOLPE AND KOENIG, P.C., UNITED PLAZA, SUITE 1600, 30 SOUTH 17TH STREET, PHILADELPHIA, PA, 19103, US

(73) Assignee: NEWTEST OY, Oulu, FI (FI)

(21) Appl. No.: 11/569,842

(22) PCT Filed: May 25, 2005
(86) PCT No.: PCT/FI2005/050175

§ 371 Date: Aug. 21, 2008

(30) Foreign Application Priority Data
Jun. 4, 2004 (FI) 20040773

(51) Int. Cl. A61B 5/103

(52) U.S. Cl. 600/595

(57) ABSTRACT

The invention relates to a method and device arrangement for determining and presenting the change of bone mass caused by accelerations having an impact on the body and representing the amount and intensity of exercise. The device arrangement according to the invention includes a transducer unit, a data transfer medium, a server and a database connected to it. The invention also relates to a transducer unit used in the device arrangement and a method utilizing the device arrangement for measuring and analyzing the intensity, amount and effectiveness of the exercise.

The invention relates to a method for determining and presenting a forecast of bone mass development caused by the exercise performance of an exercising person. The invention also relates to a transducer unit for use in the forecasting and presentation of the change of bone mass caused by the exercise performance of an exercising person. The invention also relates to a program product used in the transducer unit in the implementation of the method.

The quantity and quality of physical exercise performed by a person is known to have a significant effect on his/her state of health at present and in the future. Exercise has also been found to have an effect on osteoporosis, which is a significant national disease especially in western industrialized countries. Osteoporosis mainly occurs in the older age classes. If osteoporosis is wanted to be prevented in advance by a healthy way of life, attention must be paid to it from the childhood. Osteoporosis is a state in which a considerable amount of the mineral material contained in the bone tissue is lost. The bone may lose as much as 20% of its mass during a period of 5-7 years. When osteoporosis is far advanced, a bone embrittled by it may fracture and break as a result of even a small amount of strain. The parts that are especially susceptible to fractures are the upper part of the thigh bone and the wrist bones, which can break as a result of even a minor fall if the person has osteoporosis.

By a healthy way of life, a good diet and sufficient physical exercise stressing the supportive organs, it is possible to strengthen the bones and thus prevent osteoporosis in advance. By suitable physical exercise stressing the supportive organs, the bone mass can be increased by about 3-5%. Even with this growth of bone mass, 20-30% of bone fractures caused by osteoporosis can be avoided. According to research, the exercise performed is beneficial for the bones when sufficiently high impact on the supportive organs, such as acceleration or deceleration, or sufficiently strong torsional stress on the limb/bone, occurs during the exercise (The Lancet, vol 348, Nov. 16, 1996 pages 1343-1347). Such forms of exercise include, for example, various jumps, steps, walking, running, walking up stairs and also manual work. In order to prevent a reduction of bone mass, a person must know what kind of exercise and how much he/she should do, and also do it regularly for many years.

The published application WO 99/07280 discloses a method and an arrangement, by which the current state of the bones of a person can be examined. In the method, the person to be examined is exposed to vibration of a certain frequency for the duration of the measurement. The musculoskeletal tissue of the person is thus made to vibrate. According to the publication, this vibration of the musculoskeletal tissue also reveals the condition of the bones, i.e. whether the person has osteoporosis or not. However, the measurement of bone density according to this published application is always a one-time event, and it can only reveal the current state of the bones, and on the basis of the measurement only it is not possible to draw any conclusions on the state of the bones in the future.

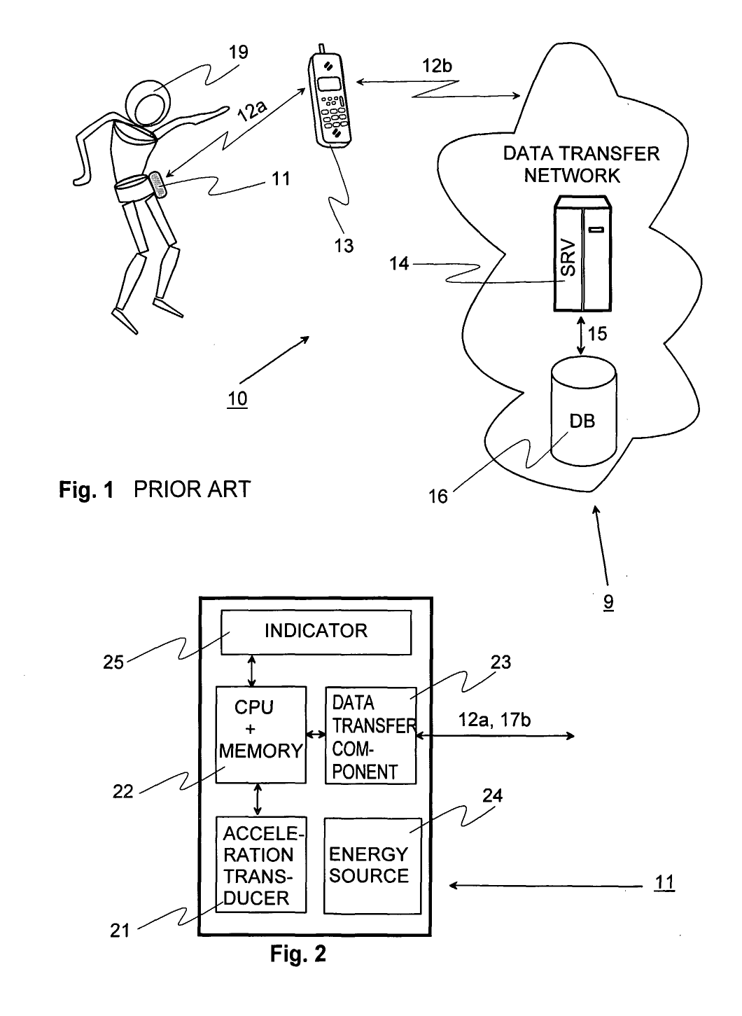

The published application WO 03/055389 of the same applicant discloses a method and an arrangement by which the state of development of a person's skeletal structure can be forecast by measuring and following up the acceleration peaks caused by the exercise the person is having. FIG. 1 shows a device arrangement according to the reference WO 03/055389 for utilizing the method. The accelerations having an impact on the person's 19 supportive organs in his/her daily activity are monitored by means of the device arrangement 10. Therefore the person 19 carries along a personal transducer unit 11, which is fastened to a belt or a corresponding piece of clothing always worn by the person.

In the solution according to FIG. 1, the transducer unit 11 measures the accelerations having an impact on the supportive organs of the person, and primarily their maximum values. The transducer unit 11 processes the measurement data by the first analysis means using an analysis algorithm according to the user's 19 requirement, and saves these results of the first analysis in the memory of the transducer unit 11. From the transducer unit 11, the saved results of the first analysis can be transferred by a data transfer connection 12a to an actual data transfer device 13. The data transfer device 13 shown in FIG. 1 is a mobile terminal device arranged to operate in a cellular network.

The analysis information can be transmitted from the data transfer device 13 by a data transfer connection 12b further to a general data transfer network 9 to be transmitted by the network to a server (SRV) 14, which is specialized in a more detailed, second analysis of the measurement results obtained from the arrangement. In the analysis, the server 14 uses the second analysis means available to it. The server 14 also comprises a connection 15 to a database (DB) 16, which contains information needed in connection with the second, more detailed analysis of the measurement data, concerning certain groups of people.

The database 16 comprises information arranged into suitable classes, such as the person's sex, age, height, weight, inherited susceptibility to a disease and the number and intensity of forces having an impact on the supportive organs per unit of time, which has been found to be beneficial according to research, based on the criteria mentioned above. In addition to that, the database 16 comprises some other pieces of information/causalities, such as tables of energy consumption known on the basis of research, and the connection between the number and intensity of forces having an impact on the supportive organs and the change of bone mass. The database 16 also comprises analysis algorithms suitable for different purposes of use, which can be transferred via a data transfer network 9 to a transducer unit 11, for example. An example of such an analysis algorithm is the analysis algorithm used for monitoring the development of bone mass presented in the reference WO 03/055389. As the basis of this analysis algorithm, it is possible to utilize pieces of research on the development of bone mass, which have been presented in the following publications, for example: Lancet, 1196, Vol 348, pages 1343-1347, Journal of Bone and Mineral research, 199, Vol 14, pages 125-128 and the publication Medicine & Science in Sports & Exercise, 2000, Vol 32, pages 1051-1057.

The bone mass development algorithm takes into account the person's basic data, the absolute values of the measured accelerations and their number during a certain measurement time. The measurement time is preferably of the order of one day, after which at the latest the algorithm is used to calculate an estimate for the person on whether the target of exercise with regard to the development of bone mass has been reached.

In comparison research, the density of the bone mass is generally measured from the heel bone or the upper part of the thigh bone. This measurement gives a general idea of the state of the person's bones. By repeated measurements, the direction of the development of the person's bones can be monitored and instructions concerning exercise or medication can be given. The method of the published application WO 03/055389 also has this idea as the starting point. However, this is a simplification which does not entirely hold good. The bone density of different parts of a person's bones can change in different ways depending on the kind of exercise and its intensity. So there is a need for a method and device arrangement, by which it is possible to forecast better than with the prior art how a certain kind of exercise and its intensity influences different parts of the bones.

It is an objective of the present invention to provide a method for measuring exercise performance and a device arrangement by which an estimate can be presented on how the exercise performed by the person influences the development of the bone mass of different parts of the bones.

The objectives of the invention are achieved by an arrangement by which information on the magnitude of the accelerations having an impact on the person's supportive organs during daily exercise is continuously collected. The measured momentary maximum values of acceleration, their shape and their number in a certain time window are saved from the measurement data. A development forecast of the bone mass of a certain part of the bones is obtained by analyzing the measurement data.

The method according to the invention for determining and presenting a forecast of the development of bone mass caused by exercise, in which method a transducer unit measuring acceleration data, carried along by the exercising person continuously measures the acceleration maximums experienced by the supportive organs of the person's body, is characterized in that the method comprises
a step in which the classified acceleration maximums are compared to reference data measured from the normal population
a step in which it is checked whether the classified results of the acceleration measurements are higher or lower than the reference data, and
a step in which an estimate of the development of bone mass is presented to the exercising person on the basis of the comparison.

The transducer unit according to the invention, which comprises a processing unit and at least one acceleration transducer, in which the transducer unit has been arranged to be used in forecasting the change of bone mass caused by the exercise performance of an exercising person, is characterized in that the processing unit has been arranged to compare the classified measurement data of the acceleration transducer of the exercising person to reference data measured from the normal population, and that the processing unit has been arranged to create an estimate of the development of bone mass from the result of the comparison, which is presented to the exercising person.

The computer program product according to the invention is characterized in that the computer program product comprises:
computer program means for classifying the acceleration maximums measured from the exercising person by an acceleration transducer into classes of acceleration
computer program means for comparing the classified acceleration maximum data to reference data measured from the normal population
computer program means for carrying out a comparison between the exercising person and the reference data on whether the classified results of the acceleration measurements of the exercising person are higher or lower than the used reference data
computer program means for creating a bone mass development forecast on the basis of the comparison performed, and
computer program means for presenting the created forecast to the exercising person.

Some preferred embodiments of the invention are presented in the dependent claims.

The basic idea of the invention is the following: The person carries along a light transducer unit, which measures and registers the accelerations experienced by the person during his/her daily or training exercise that have an impact on the supportive organs. In this connection, acceleration means either an increase of the speed of movement or a reduction thereof, in which case the latter is often also called deceleration. The momentary maximum values, the shape of the acceleration peaks and their number in a certain time window are measured from the accelerations. On the basis of the acceleration data measured in a certain time window, a forecast is calculated for the development of the bone mass of a certain part of the skeletal structure. If the person wants to increase the bone mass in a certain part of the skeletal structure, the analysis can be performed by parameters that best describe the desired part of the skeletal structure. When required, the transducer unit can present the results of the first analysis made by it. The acceleration measurement data are saved at least temporarily in the electronic part of the transducer unit. The method and device arrangement according to the invention are intended to guide the person to perform the right kind of exercise with regard to the personal objective.

The invention has the advantage that from the measurement data of the accelerations experienced by the person during exercise, which have an impact on the supportive organs, it is possible to create a forecast for the development of the bone mass of a certain part of the skeletal structure.

In addition, the invention has the advantage that the person can, when he/she so wishes, have up-to-date information available, on the basis of which the amount and intensity of exercise can be monitored and controlled in view of a certain part of the skeletal structure.

The invention also has the advantage that all the time the person has available the amounts of performance for different kinds of exercise, which can be used when drawing up a personal recommendation for additional exercise.

In the following, the invention will be described in more detail. Reference will be made to the accompanying figures, in which

FIG. 1 shows the parts of a device arrangement used in the estimation of bone mass according to the prior art by way of example,

FIG. 2 shows, as an example, the transducer unit of the device arrangement according to the invention, which is carried along by the person,

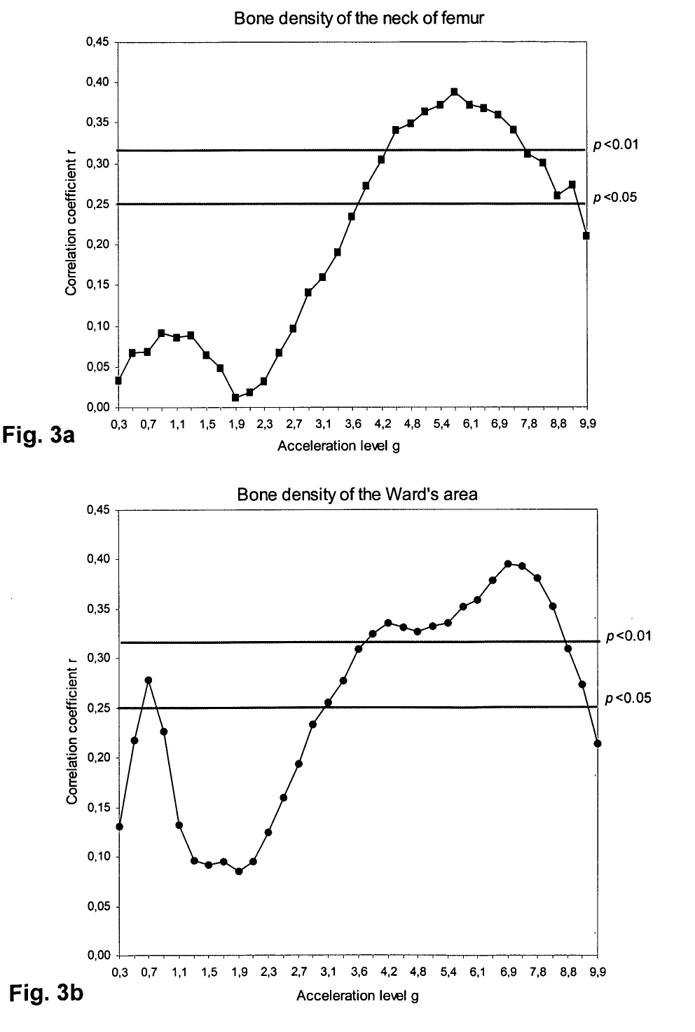

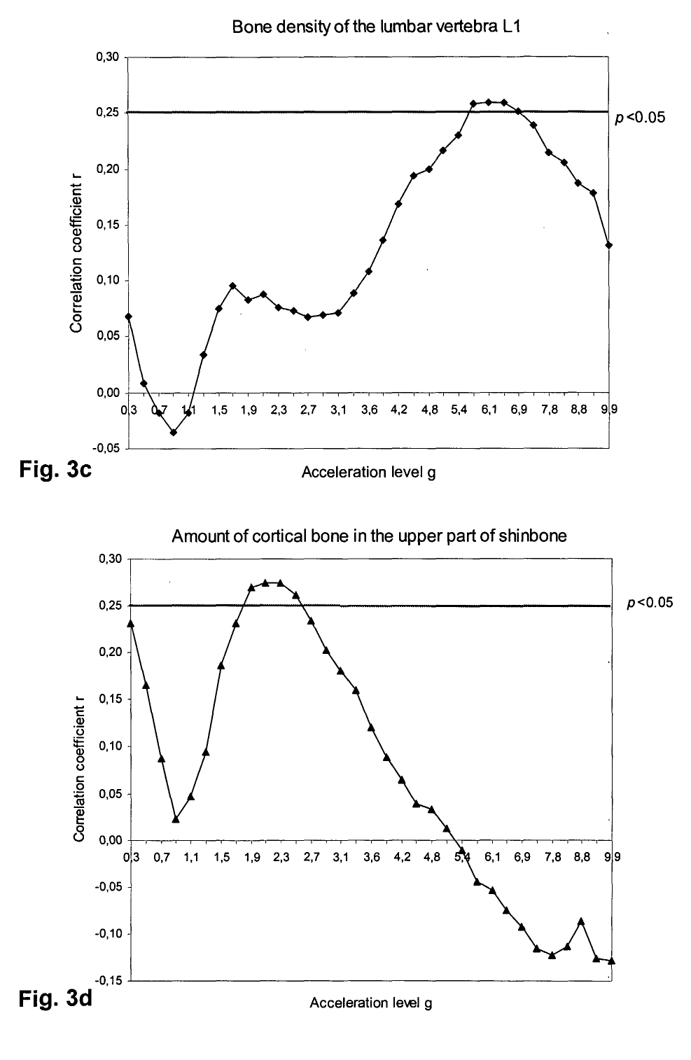

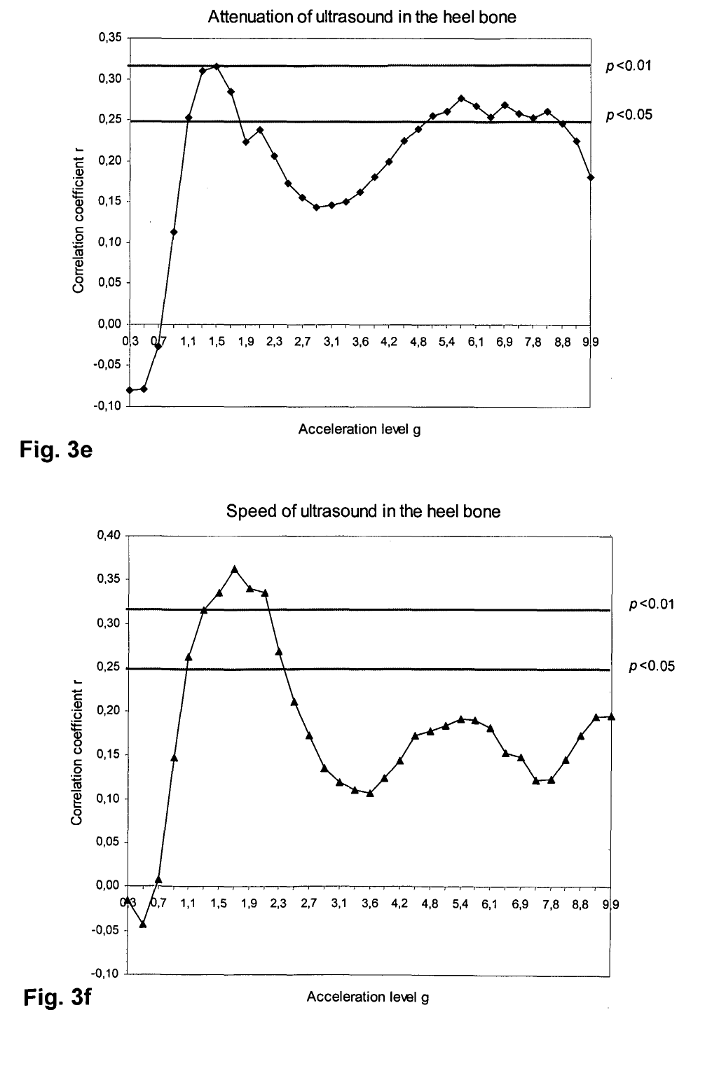

FIGS. 3 a-f present the relationships between the measurement results of accelerations and the development of bone mass in different parts of the skeletal structure, and

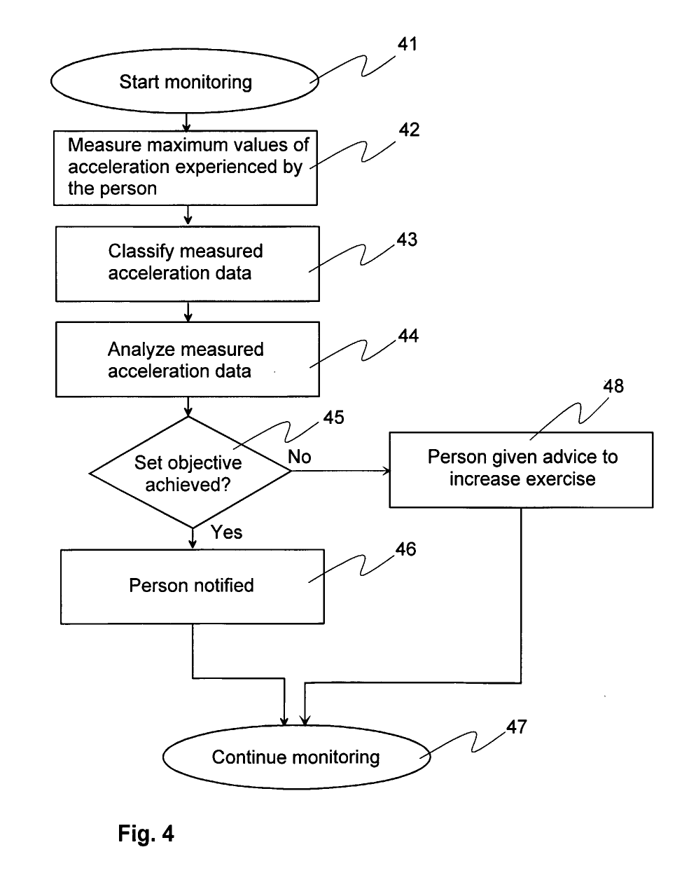

FIG. 4 shows, as an exemplary flow chart, a method for measuring and analyzing the accelerations having an impact on the supportive organs, which utilizes the device arrangement according to the invention.

FIG. 1 was explained in connection with the description of the prior art.

FIG. 2 shows a preferred embodiment of the transducer unit 11 according to the invention. The transducer unit 11 preferably includes an energy source 24, such as a battery or accumulator. The electric elements contained by the transducer unit 11 get the energy required for their operation from this energy source 24. There is at least one acceleration transducer 21 in the transducer unit 11. By using more than one transducer 21, the accelerations can be measured in two or three dimensions, when required. The range of measurement of a single acceleration transducer is advantageously ±12 g.

The accelerations measured in the transducer unit 11 can be analyzed in different ways. Some exemplary, advantageous ways have been presented in connection with the description of FIG. 3. One possible way of estimating the development of bone mass is to use the number (N) of the times of observation of the acceleration maximums measured at a certain level of acceleration in a suitable time window. The width of the class of acceleration level used in the measurement is advantageously of the order of 0.3 g. If the time window is relatively short, some seconds, it can also be found out from the acceleration measurement data produced by the transducer unit 11 what kind of exercise the person has performed. This piece of information can also be utilized when estimating how the exercise has developed the bone mass in different parts of the skeletal structure. On the basis of this information, the person can be given a suitable recommendation for additional exercise.

The measurement data of the acceleration transducer 21 is transmitted to the Central Processing Unit (CPU) of the transducer unit 11, which also advantageously comprises a certain amount of memory used by the processing unit 22 for saving various program applications and the results of performed acceleration measurements according to the invention. The processing unit 22 advantageously performs an analysis of the measured acceleration data by the first analysis means saved in its memory, suitable for a certain piece of training.

The processing unit 22 is also connected to a data transfer component 23. By this data transfer component, a data transfer connection can be set up to a terminal device 13 of a cellular network, connection 12a. The data transfer component 23 advantageously supports at least one data transfer method. Advantageous methods used in data transfer include infrared (IR), Bluetooth, WLAN and various time or code division data transfer techniques used in cellular networks.

The transducer unit 11 advantageously also comprises indicator means 25 for indicating the result of the first analysis specific to the time of use, for example. By the indicator means it is possible to communicate to the user of the transducer unit 11 as simple messages of the ON/OFF type, for example, whether the exercise performed meets the set objective, considering a certain period of time. The indicator means 25 can be LEDs of different colours, for example. If the set objective of the exercise performance is reached, a LED of a certain colour burns. Alternatively, the indicator means 25 may comprise a bar-shaped display, the length or lengthening of which shows the result of the exercise performance compared to the set objectives. The information presented by the indicator means 25 can be either specific to the time of use or cumulated from a certain period of time. The user can advantageously modify the way in which the information is presented to make it support his/her personal exercise best.

The transducer unit 11 according to the invention can also be part of some other device, which the person being monitored carries along. An example of such a device, to which the transducer unit 11 according to the invention can be integrated, is a terminal device (13) used in cellular networks. In that case, at least one acceleration transducer and a program application implementing the method steps according to the invention have been installed in the terminal device (13) to provide the operation according to the invention.

When required, the actual, more accurate analysis can be performed later at a desired time in a server 14, for example, on the basis of data transferred to its database 16 from the transducer 11. The analysis performed in the server 14 gives a more detailed picture of reaching the objective set by the person, because history data describing the person's exercise saved earlier in the server 14 are also used for making the analysis.

Table 1 and the FIGS. 3a-f related to it illustrate by means of graphs the correlation between the measured accelerations and the development of bone mass in a certain part of the skeletal structure, which research has found to exist. The limits of statistical significance p<0.05 (statistically significant) and p<0.01 (statistically very significant) have also been drawn in FIGS. 3a-f.

Table 1 is based 80 on a 12-month follow-up carried out on a female (age 35-40). Half of the target group was guided to intensified exercise and the other half operated as a reference group. Table 1 is divided into four different ranges of acceleration 0.3-1.7 g, 1.9-3.3 g, 3.6-5.7 g and 6.1-9.3 g. In addition, each range of acceleration has also been divided into measurement classes of approximately 0.3 g. The connection between exercise activity and the development of bone mass has been calculated by linear regression analysis.

TABLE 1
The effect of exercise on bone density:

| Object of | | | | |
|---|---|---|---|---|
| measurement | 0.3-1.7 | 1.9-3.3 | 3.6-5.7 | 6.1-9.3 |
| Neck of femur | ns | ns | 0.391** | 0.394** |
| Trochanter | ns | ns | 0.410** | 0.451** |
| Ward's area | ns | ns | 0.356* | 0.414** |

The term “ns” in Table 1 means that no correlation has been found, the marking * means a statistically significant correlation (p<0.05) and ** means a very significant correlation (p<0.01).

FIGS. 3a-f show examples of correlation graphs drawn from the information of the same piece of research from different parts of the skeletal structure. The graphs have been obtained by proportioning the numbers (N) of acceleration peaks included in different acceleration levels experienced by the test persons to an average value measured from a control group.

FIG. 3a shows the correlation of the development of bone density measured from the neck of the thigh bone to the relative numbers of measured acceleration pulses at different levels of acceleration. FIG. 3b shows a corresponding graph as measured from the so-called Ward's area of the thigh bone, and FIG. 3c shows the corresponding correlation graph of the lumbar vertebra L1. FIG. 3d presents a corresponding graph for the amount of cortical bone in the upper part of the shinbone. FIGS. 3e and 3f present the corresponding graph of the heel bone as measured by two different methods of measurement. In FIG. 3e, the attenuation of ultrasound in the heel bone has been used, and the speed of ultrasound in the heel bone has been used in the measuring method of FIG. 3f.

On the basis of table 1 and the FIGS. 3a-f it can be generally stated that in the range of low acceleration, under 2 g, no clear correlation between the acceleration measurements and the development of bone mass can be found. It seems that bone mass is developed in the heel bone and the shinbone already with relatively low acceleration values (under 2 g), whereas clearly higher acceleration maximum values (3-9 g) are needed at the upper part of the thigh bone and the lumbar vertebra, in order to achieve development in the desired direction.

Accelerations that can be classified as having a low acceleration level (under 2 g) are created in a person's normal, daily exercise. A person practicing intensified exercise therefore mainly needs information on whether accelerations having a higher acceleration level have been achieved or not. In that case, mainly information on measurement values occurring in the acceleration range 3-9 g is needed. When this measurement result is compared to the average measurement result of the population, a forecast can be calculated from this comparison for the person on whom the measurement is carried out on the development of his/her bone mass.

The comparison of the person's exercise to the reference population can be carried out in the following ways, for example. One possibility is to make a direct comparison between the measurement results of the person, the number of acceleration pulses (N) by classes of acceleration level, and the corresponding measurement results of people of the same sex and age. This reference group does not practise intensified exercise.

The measured acceleration peaks can also be analyzed on the basis of their shape. In that case, the factors that can be utilized in the estimation include, in addition to the mere number of peaks (N) or instead of it, the width of the acceleration peak, the area of the acceleration peak, the area of the acceleration peak squared, the ascending time of the acceleration peak or the ascending angle of the acceleration peak. The accuracy of the bone mass forecast can be increased by using these parameters.

Another possible way of making a forecast on the development of bone mass is to calculate the area defined by the acceleration level graph drawn up from the measurement results preferably in the range 3-9 g. In this graph, the variables are the acceleration maximum classes (x-axis) and the relative number of the occurrences of a certain acceleration maximum class (y-axis). The area calculated from the graph is then compared to the area obtained from the measurements of the corresponding normal population. If the area calculated from the graph of the exercising person exceeds a certain threshold value (the result of the normal population), the exercise performed has an increasing effect on the bone mass.

The third possible way of forecasting is to convert the measured numbers (N) of the acceleration peaks in a certain acceleration level class to logarithmic (log(N)), in which case the straight line y=ax+b can be mathematically fitted as the graph of acceleration level classes. The constants a and b are compared to a straight line, which has been obtained from the measurement results of normal population of the same age and sex. The normal population does not practise intensified exercise. If the constants a and b of the person being monitored exceed certain threshold values calculated on the basis of the normal population, the exercise performed by the person has had a positive effect on the development of bone mass.

FIG. 4 shows the main steps included in the method according to the invention as an exemplary flow chart. The facts explained in connection with FIGS. 1-3f are also utilized in connection with the description. By the method it is possible to create an indication as to whether the set objective for the development of bone mass has been achieved by the person's exercise or not.

The collection of measurement information on accelerations is started in step 41. In principle, the measurement is continuously in operation after this. This is possible because the person 19 being measured equips himself/herself with a trans-ducer unit 11 according to the invention. In step 42, while the person is exercising, the transducer unit 11 continuously collects various information on the accelerations experienced by the person, their absolute values, the shape of the acceleration pulses, the frequency of occurrence and number.

In step 43, the measured acceleration data are classified. The classification of acceleration data according to the invention can be carried out either during the whole measurement or after a certain time. The classification of acceleration data of acceleration maximums can be carried out on the basis of the acceleration level measured from the maximum, for example. In that case, 0.3 g can advantageously be used as the class interval. The number (N) of acceleration maximums occurring in a certain acceleration range is found out by this procedure, and is then used for drawing up an estimate of the development of bone mass.

In addition to or instead of the classification described above, the width of the measured acceleration peaks, the area of the accelerations peaks, the area of the acceleration peaks squared, the ascending time of the acceleration peaks or the ascending angle of the acceleration peaks can be utilized in the classification, when required.

In step 43, the classified acceleration data, examples of which have been pre-sented in connection with FIGS. 3a-f, are processed with a suitable analysis algorithm. The result of the analysis made is compared in step 44 to suitable reference data, which have been obtained by examining the normal population. If the result of the comparison shows that exercise has been sufficient, the person is notified of that. The notification can be either a simple notice of the ON/OFF type, or it can also be given as a message transmitted by a communication device in the form of sound, text or image.

If the comparison in step 45 gives a negative result, it means that the exercise activities of the person have not been sufficient. Then the person is given advice in step 48 to increase exercise. This advice 48 can advantageously also contain an example of what kind of exercise and how much would be necessary. This is possible, because certain forms of exercise are known to have a different impact on the bones.

Regardless of the result of the comparison in step 45, the collection of acceleration measurement data continues in step 47. In practice, the process returns back to step 42. The repetition rate of step 45 can be decided personally by the person being measured. It may take place by a few hours, daily, weekly or monthly. This time may also be determined on the basis of the size of the memory 22 of the transducer unit 11 carried along by the person. When the memory 22 of the trans-ducer unit 11 is filling up, the comparison 45 should be carried out at the latest. After this, the results of the comparison 45 can be transferred via a suitable data transfer connection 12a, 12b to a device having a higher data processing capacity, such as a server 14 and in it a database 16, to which personal exercise information is gathered by this procedure. On the basis of this information, a new, more accurate estimate on the development of the person's bone mass during a longer period of time can be drawn up.

The more detailed, second analysis of the measurement data is thus preferably carried out in the server 14 by the second analysis means. They utilize both the personal data and general data concerning certain groups of people saved in the database 16. The personal data include the analysis results of all the previously saved exercise data. If this more detailed analysis shows that it would be advantageous for the person to perform certain kind of additional exercise in order to reach his/her own objective, a recommendation on this can be drawn up for him/her.

The method steps according to the invention described above can advantageously be implemented by two separate program applications. The first program application has been saved in a transducer unit (11) and the second one in a server (14). The first program application saved in the transducer unit (11) can also be utilized as separate. If long-term monitoring of the exercising person is wanted to be implemented, another program application in the server (14) is also needed.

Some preferred embodiments of the invention have been described above. The invention is not limited to the solutions described above. The objects of measurement of acceleration data presented in the description are only examples of the objects in which the method according to the invention may be applied. The inventive idea can be applied in numerous ways within the scope defined by the attached claims.

1. A method for determining and presenting a forecast of the bone mass development caused by the exercise performance of an exercising person (19), concerning one part of the skeletal structure, which method comprises:
a step (42), in which a transducer unit (11) measuring acceleration data, carried along by the exercising person (19), continuously measures the acceleration maximums experienced by the supportive organs of the body of the exercising person,
characterized in that for drawing up an estimate concerning one part of the skeletal structure, the method further comprises:
a step (43), in which the acceleration maximums measured by the transducer unit (11) from a certain period of time are classified as belonging to an acceleration level class, where the classification comprises:
defining before measurement an upper and lower limit for an acceleration measurements range based on one part of the skeleton structure;
dividing the defined acceleration measurements range in equal acceleration level classes; and
classifying during measurement each measured acceleration maximum to a certain acceleration level class, in which case a classified maximum adds occurrence number (N) by one in that acceleration level class;
a step (44), in which the classified acceleration maximums are compared to reference data measured from normal population;
a step (45), in which it is checked if the classified acceleration measurement level classes of the exercising person (19) are higher or lower than the reference data, and
a step (46, 48) in which an estimate of the bone mass development concerning one part of the skeletal structure is presented to the exercising person (19) on the basis of the comparison.

2. The method according to claim 1, characterized in that a measuring range for acceleration maximums is between +0.3 g and +12 g and that the width of one acceleration level class belonging to it is of the order of 0.3 g.

3. The method according to claim 1, characterized in that the numbers of occurrence (N) included in selected acceleration level classes are compared to corresponding numbers of occurrence measured from the normal population, and that a forecast for the development of bone mass of one part of the skeletal structure of the exercising person (19) is calculated from the result of the comparison.

4. (canceled)

4. The method according to claim 1, characterized in that a graph is formed of the gathered numbers of occurrence (N), with the acceleration classes as the argument and the relative numbers of occurrence of the acceleration level classes as the function, and that the area between two acceleration levels defined by the graph of the function is determined and that the area determined is compared to the corresponding area obtained from measurements of the normal population, and that the result of the comparison is used to draw up a forecast of the development of the bone mass of one part of the skeletal structure of the exercising person (19).

5. The method according to claim 1, characterized in that a graph is formed of the gathered numbers of occurrence (N) belonging to different acceleration level classes, with the acceleration level classes as the argument and the logarithm (log N)) of the numbers of occurrence of the acceleration level classes as the function, which logarithmic function is fitted to a straight line y=ax+b, the constants a and b of which function are compared to corresponding constants a and b of a corresponding straight line obtained from the measurements of the normal population, and that a forecast of the development of the bone mass of one part of the skeletal structure of the exercising person (19) is created from the result of the comparison.

6. A transducer unit for use (11) in forecasting a change of bone mass caused by the exercise performance of an exercising person (19) in one part of the skeletal structure, which transducer unit comprises:
at least one acceleration transducer (21) for measuring the acceleration maximums experienced by the person as a continuous operation, characterized in that the transducer unit further comprises:
a means belonging to a processing unit (22) of the transducer unit for classifying measured acceleration maximums to acceleration level classes, which classifying means are arranged to:
define before measurement an upper and lower limit for an acceleration measurements range based on one part of the skeleton structure;
divide the defined acceleration measurements range in equal acceleration level classes, and; classify during measurement each measured acceleration maximum to a certain acceleration level class, in which case a classified maximum adds occurrence number (N) by one in that acceleration level class;
a means for comparing the classified acceleration maximums of the exercising person (19) to reference data measured from the normal population;
a means for creating from the result of the comparison an estimate on the development of bone mass concerning at least one part of the skeletal structure, which is presented to the exercising person (19).

7. The transducer unit according to claim 6, characterized in that the measuring range for acceleration maximums is between +0.3 g and +12 g and that the width of one acceleration level class belonging to it is of the order of 0.3 g.

8. The transducer unit according to claim 6, characterized in that the numbers of occurrence (N) of acceleration maximums included in the selected acceleration level classes have been arranged to be compared to corresponding numbers of occurrence measured from the normal population, and that a forecast for the development of the bone mass of one part of the skeletal structure of the exercising person (19) has been arranged to be calculated from the result of the comparison.

10. (canceled)

9. The transducer unit according to claim 6, characterized in that a graph has been arranged to be formed of the gathered numbers of occurrence (N), with the acceleration classes as the argument and the numbers of occurrence (N) of the acceleration level classes as the function, and that the area between two acceleration levels defined by the graph of the function has been arranged to be determined and that the area determined has been arranged to be compared to the corresponding area obtained from measurements of the normal population, and that a forecast on the development of bone mass of one part of the skeletal structure of the exercising person (19) has been arranged to be calculated from the result of the comparison.

10. The transducer unit according to claim 6, characterized in that a graph has been arranged to be formed of the gathered numbers of occurrence (N) belonging to different acceleration level classes, with the acceleration level classes as the argument and the logarithm (log N)) of the numbers of occurrence of the acceleration level classes as the function, which logarithmic function has been arranged to be fitted to a straight line y=ax+b, the constants a and b of which function have been arranged to be compared to corresponding constants a and b obtained from the measurements of the normal population, and that a forecast on the development of the bone mass of one part of the skeletal structure of the exercising person (19) has been arranged to be calculated from the result of the forecast.

11. The transducer unit according to claim 6, characterized in that the estimate of the development of bone mass concerning one part of the skeletal structure has been arranged to be indicated by indicator means (25) belonging to the transducer unit either as specific to the time of use or as a cumulative estimate.

12. The transducer unit according to claim 11, characterized in that the indicator means comprise an indicator of the ON-OFF type, in which the ON state indicates that the target specific to the time of use has been reached, and the OFF state indicates that the target of the exercise performance has not been reached.

13. The transducer unit according to claim 12, characterized in that the indicator (25) has been implemented by two LEDs, one of which indicates the ON state when burning and the other one indicates the OFF state when burning.

14. The transducer unit according to claim 12, characterized in that a pointer arrangement, which has been arranged as growing according to the exercise performance, has been arranged to be used as the indicator means (25).

15. The transducer unit according to claim 6, characterized in that the transducer unit (11) is part of a terminal device (13) of a cellular phone network.

16. A computer program product on a data transfer medium for creating and presenting an estimate of the development of bone mass concerning one part of the skeletal structure, characterized in that the computer program product comprises:
computer program means for classifying acceleration maximums measured by an acceleration transducer (21) from an exercising person (19) into acceleration level classes, which computer program means includes:
a means for defining before measurement an upper and lower limit for an acceleration measurements range based on one part of the skeleton structure;
a means for dividing the defined acceleration measurements range in equal acceleration level classes, and;
a means for classifying during measurement each measured acceleration maximum to a certain acceleration level class, in which case a classified maximum adds occurrence number (N) by one in that acceleration level class;
computer program means for comparing the classified acceleration maximum data to reference data measured from the normal population;
computer program means for carrying out a comparison between the exercising person and the reference data on whether the classified acceleration measurement results of the exercising person are higher or smaller than the reference data used;
computer program means for creating a forecast on the development of bone mass concerning one part of the skeletal structure on the basis of the comparison; and
computer program means for presenting the forecast to the exercising person.
